(19) United States
(12) Patent Application Publication
(10) Pub. No.: US 2002/0120310 A1
(43) Pub. Date: Aug. 29, 2002

(54) Medical device systems implemented network system for remote patient management

(76) Inventors: Gregory J. Linden, Shorewood, MN (US); Kenneth M. Riff, Orono, MN (US)

Correspondence Address: Girma Wolde-Michael, Medtronic, Inc., MS 301, 7000 Central Avenue N.E., Minneapolis, MN, 55432, US

(21) Appl. No.: 09/935,019

(22) Filed: Aug. 22, 2001

Related U.S. Application Data
(60) Provisional application No. 60/227,164, filed on Aug. 22, 2000.

(51) Int. Cl. A61N 1/08

(52) U.S. Cl. 607/60; 600/300

(57) ABSTRACT

A system and method for computer enabled network patient management of medical devices used in chronic disease management. Utilizing web site and push alert notification of alert level physiologic data derived via analysis of continuous stream wireless data transmissions from a patient, a full cycle improvement over existing modalities is achieved. Proxy and medical device user integration and access is enabled to achieve further contribution to the technical advantages of the system.

### FIELD OF THE INVENTION

The invention relates to medical devices implemented and communicable through network systems such as the internet. More specifically, the invention relates to patients wearing implantable or externally mounted medical devices in which the devices are configured to be communicable with remote healthcare professionals and other remote patient monitoring systems.

### BACKGROUND OF THE INVENTION

Medical devices such as cardiac systems, drug delivery systems, neurological products and similar other products are implanted in patients for various clinical reasons. Some of these devices may collect and document data on a continuous basis. However, the state of the art is currently to ask patients to see their doctors or other health professionals on a regular basis to retrieve and check the physiological data collected in these devices.

As medical devices become very sophisticated, in both reliability and maintainability, the need for patients to visit their doctors on a regular basis may not be required by coverage plans or for other rationale. Various attempts to remotely engage or monitor patients have been suggested, such as for example those described in U.S. Pat. Nos. 6,168,563 B1; 6,221,011 B1; 6,203,495 B1; 6,250,309 B1; 5,633,910 and 5,752,976. However, for patients with chronic disease, the management of the disease has become a critical aspect which affects both cost and the quality of life of the patient. Accordingly, patients with implantable medical devices or externally mounted devices that monitor critical medical or physiological data are either kept in hospitals or the patients are required to visit their physicians on a very regular basis. However, a data transfer and review system that enables doctors and physicians to monitor patients on an as-needed basis and as frequently as possible, while allowing patients to stay at home, is a highly desirable service. Such a service would also enable the patient to have access to their own personal data by enabling real time data management and review by professionals as well as the patient. Further, medical devices could be designed to enable patients to be interactive with the devices that are monitoring their physical and medical parameters such that the patients could also be involved in managing their disease on a day-to-day basis. More specifically, if patients are allowed to have access both to the operation of their device and reports that are stored in them, they may have sessions with their doctors and will also be well-informed in managing their disease, thereby becoming active partners in the management of their own chronic disease.

### SUMMARY OF THE INVENTION

An internet-based system and method for a service is disclosed which connects a remote patient to a database network for medical device data exchange and review. The system and method includes providing a web-site having a user interface wherein the user interface includes a secure sign-in input to access a medical device database network site, receiving at the web-site inputs associated with a specific medical device and patient, confirming the identity of the medical device and the patient, and enabling the patient to access the database to use the service. A further aspect of this system and method of doing business is to connect a remote patient's friends and family to a database network for medical device exchange and review by identifying friends and family authorized to access the database network and/or sending notification of a patient alert to said friends and family so that they may be involved in the patient's care.

### BRIEF DESCRIPTION OF THE DRAWINGS

FIG. 1 is a schematic representation of a patient with exemplar implanted medical device components interfacing with remote patient management network systems.

FIG. 2 is a schematic diagram of the system and data flow of the invention.

FIG. 3 is a schematic diagram of the system and data flow of the invention.

FIG. 4 is a schematic diagram of the system and data flow of the invention.

FIG. 5 is a decision making flow chart of the method of the invention.

### DESCRIPTION OF THE INVENTION

Attempts to provide improved healthcare to patient populations using technological methods have met with varying degrees of success. Indeed, however, a very critical problem has emerged in most such efforts. The patients and healthcare providers alike risk increased isolation as the use of technology increases. This isolation and sense of de-personalization in the healthcare system is often a chief complaint of patients, as well as a source of potential conflict which could actually impede the formation of candid dialogue which is at the core of the best models of patient-physician interface.

Assignee of the Applicants (Medtronic, Inc.) has developed medical devices which are able to detect large amounts of valuable patient specific information and process that information so as to decide whether one or more specific applications of therapy or ongoing diagnosis is merited. This is technology that has emerged in application of implantable medical devices over the last several decades, and has transformed many lives due to its use by Medtronic and other companies. However, Applicants have now identified new and improved mechanisms by which the technical means of harnessing the ongoing and simultaneous revolutions in medical device technologies and information technologies to achieve a new level of patient and healthcare provider satisfaction, quality of care, improved service, improved efficiencies and improved economics is realized. This combination of new technical processes has overcome the combined technical problems associated with: sensing highly specific signals, parsing and validating high volumes of data, power management of implanted medical devices, bandwidth allocation to integrate all levels of a web-enabled network among multiple foreign users, timing of signals and processing, accessibility and security, display limitations and demands, data routing in multiple paths including wireless paths, partial user unfamiliarity with technology, partial user incapacity, and other challenges. The technical effect of the selected protocols along with the application of advanced information technology has resulted in a significant technical contribution to the art of integrated network-based signal and resource management for programmable and high relevance detected data signals.

Applicants have recognized that a remarkable innovation is achieved in deeply utilizing the now very robust data collection capabilities of various medical devices and integrating that information (either before, during or after analyzing or processing the information) into a data network. This causes integration of the patient and healthcare providers, or others, into a collaborative effort resulting in patient-specific healthcare improvement and system efficiencies. This is a pioneering effort in identifying a combination of historic problems, applying technical methodology to combine the best medical and information technologies resulting in a solution which transforms the way resources are allocated, which in turn leads to improvements for care of patients with various diseases and chronic medical conditions, particularly those having chronic conditions.

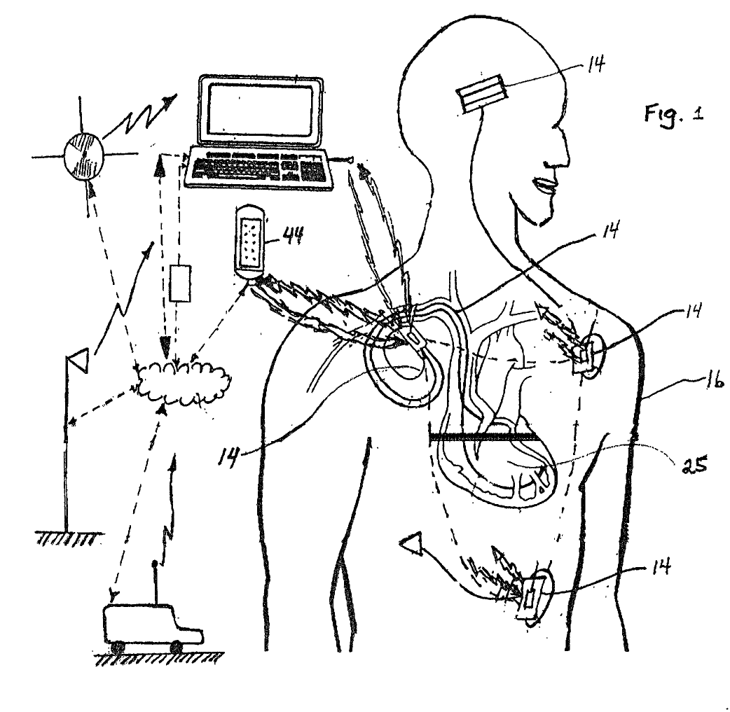

In one example, patients having advanced cardiovascular disease (and their care givers) may utilize this invention to great advantage. Using one or more medical devices 14, such as the implantable cardiac-related devices known as either the Medtronic Chronicle device or the GEM III DR device, shown in patient 16 of FIG. 1, a patient's cardiac system is closely monitored. This figure also illustrates alternate examples of device 14 which includes neurologic device 14 and drug infusion device 14 as well. The invention may utilize a device such as those noted above which is implanted for sensing directly in a cardiac chamber 25, and which may have more data acquisition capability and processing power than any other known implantable on earth, or even a more limited capability device. Such a powerful data acquisition platform is able to store a wealth of information regarding cardiac and overall patient physiologic data in the onboard RAM of the device that can be uploaded through wireless technology, such as radio frequency (RF) telemetry, telemetry C, or the like, and which is representatively shown within lines 31 of FIG. 1. Following acquisition of this medical data from inside the body, then advanced information and communication technologies are utilized to communicate this information to other locations or users.

In one embodiment, another device, shown in representative manner as device 44 in FIG. 1, is arranged to be near the patient and to obtain the information from the implanted device and transmit the data to a remote network. Device 44 may be alternately described as a remote interrogator or by other terminology, which is not meant to limit its actual technical characteristics in any way, but rather than to identify a data communication device for use with medical devices as generally described herein.

As shown in FIGS. 1-5, a secure transmission of the physiologic and medical data from the patient then occurs, with the data flowing to a robust network where it can be stored, processed, analyzed and presented for viewing via a web browser or other interfaces. These technologies have been combined and improved with the specific purpose of providing a reliable, scalable, secure and accessible system for worldwide real-time use of the patient's data. This is an extraordinary, full cycle, breakthrough over known limited systems for disease and patient management. Indeed, this invention re-organizes and prioritizes patient care in a manner not previously known along a new model of data and human interaction. In the cardiac example above, information about the patient's heart is online and accessible at all times. The system provides the physician and others with a continuous, longitudinal record of cardiac status rather than the snapshot that is received when the patient comes into a clinic. This is also real-time and continuous, rather than responsive and subject to any specific event or to known monitoring device availability or limitations. Physicians may then use this new system and methods to implement timely, systematic therapeutic regimens for their patients. It is recognized that either the virtual or the physical locations of certain elements of this system, and methods of performing the services and advantageous steps of the invention, may be optionally determined to occur in either one or more jurisdictions depending on the element, step, signal or advantage enabled. Accordingly, appropriate data use and other security authorizations or measures are fully contemplated, and may be relevant to judging the utilization and scope of challenges successfully overcome by this invention.

Using this invention, the patient, and optionally the patient's friends and family, become active participants in the management of the patient's health. This enables the patient to be empowered as a collaborator through the system's patient portal, where he or she can access individualized educational, monitoring and selfcare programs at any time from virtually any location. Although a deep and rich variety of data is acquirable, data having the most relevance for the specific patient may be designated for sensing, e.g. a patient's monitored signals may include intracardiac pressures, heart rate, physical activity, or other signals. This process of continuous data collection is then supplemented by the uploads of all or some of the data to an external proximate device (i.e. in home, office or car) for transmission of the critical physiologic data securely to a healthcare management network. Physicians can access the network via a Web site at any time and review screens that present summary information from the latest upload, trend information accumulated over time, and/or detailed records from specified times or problem episodes.

The invention has the potential to transform the management of chronic diseases like heart failure and cardiac arrhythmias, neurologic conditions, or conditions requiring drug infusions, and other health needs relating to health conditions. It will improve the quality of care by providing the physicians with real-time data about their patients' physiological (and maybe other) conditions so that corrective actions may be taken in a timely manner, if appropriate. This invention will improve the quality of life for the patients, enabling them to remain in their homes or travel as desired without the same level of constant worry concerning an emergency hospitalization. The invention will result in dramatic lowering of the cost of managing chronic disease by reducing unnecessary hospitalizations and clinic visits. Perhaps most importantly, this system and methodology will help restore the close bond of the patient-physician relationship through the power of direct connectedness and the proliferation of high relevance understandable knowledge to all participants in the process. For example, the system and methodology makes personalized health information, even detailed information on the performance of a patient's own heart, available to the patient and loved ones (or other advocates of the patient) through an interactive web or mobile portal. Trials of this system have already observed these very positive effects, synergies and impacts.

In addition to the profound implications for quality of care, standard of care, and related issues in relation to this invention, the financial and business impact of this system and methodology is quite revolutionary. The system is designed to more successfully manage people with chronic diseases by leveraging the Internet or similar communication medium that allows continuous or near continuous real-time data access. The interaction with clinical researchers of users of this invention is also valuable. This degree of interaction allows for more rapid and in-depth clinical analysis of disease symptoms and treatments, thereby reducing the overall costs of such efforts. Such real-time access to patient data also facilitates administration of drug and other therapy in a more responsive and economical fashion. The involvement of the patient and the patient's advocate(s) promotes improved communication and outcomes. Combining this with the known patient desire for greater convenience and a closer connection to the physician, then the advantages of this invention are many.

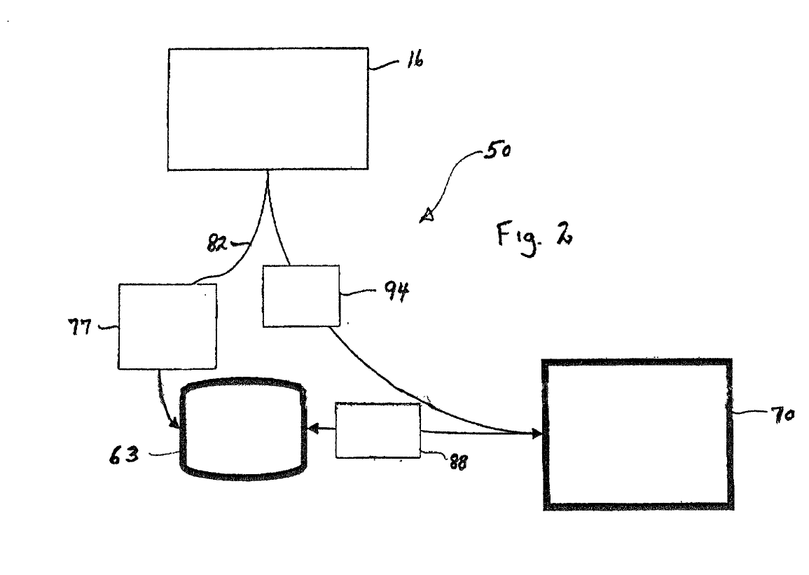

FIG. 2 is a schematic representation of a portion of system 50 designed for interacting and managing care for a patient 16 having at least one interactive medical device, such as for example an implanted cardiac, neurologic, infusion or other device. It is recognized that partially implanted or external devices may also be incorporated into the full scope of this disclosure, provided such devices or components are capable of operating in the robust data environment of this invention. FIG. 2 illustrates the relation between patient 16, an electronically accessible patient management database 63, and a web-based site 70. Patient management database 63 is configured for receipt, storage, processing, and other transmission and handling of information related to the healthcare status of patients administered by system 50, including patient 16. Physiologic data 77 is uploaded via wireless data communication medium 82 or other transmission means from devices in or in communication with devices in patient 16, and is of a content designed to provide essential high relevance information regarding all manner of monitored disease etiology. This information is automatically uploaded via path 82 to the database 63, possibly in response to interrogation routines, and subject to power management, disease stage, and other considerations. Data 77 is then registered or otherwise processed as appropriate and transmitted in various form to at least one web-based site 70, configured to allow secure sign-in access by the patient and others, as will be further discussed below, and as depicted by box 94 with one or more secure sign-in protocols included. It is recognized that patient medical device data 88 may also flow from web-based site 70 back to patient management database 63.

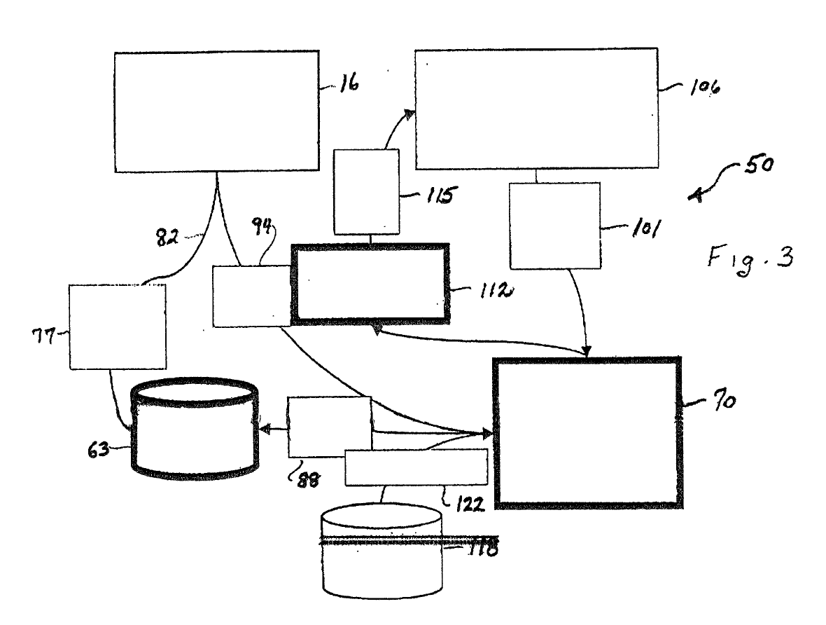

FIG. 3 depicts web-based site 70 as a platform which is accessible via secure sign-in 101 by a first medical provider 106 such as a nurse or other type of provider responsible for the care and first stage monitoring of patient 16. A variety of interactions are enabled by this relationship, including use of web-based site 70 as a destination for secure access to provide and receive information pertinent to the care of patient 16, including unique and high relevance physiologic data 77 detected from within one or more patients and transmitted via patient management database 63. In the event of physiologic data 77 containing information meeting certain criteria, such as activating triggers or set points based upon certain analytics, differentials or algorithmic metrics, then the patient management database 63 and the web-based site 70 may be configured to provide an automatic event notification service signal 112 to a display or other data receiving device of one or more medical providers, such as a first medical provider 106. In one example, automatic event notification service signal 112 may be pushed via an SMTP.net message, shown for example as signal format 115, to medical provider 106 computer or other display. These displays, and others contemplated herein, may also include an automatic pushed signal via electronic mail, pager, cellular phone, WAP cellular phone, telephone call, facsimile, mobile wireless device, stylus tablet, or others. This automatic event notification service signal 112 may be used to alert provider 106 of a health related signal anomaly or other indication which may require prompt action to assist the patient, and is likely not necessarily a secure message but rather a rapid message is preferable. Accordingly, in one embodiment, the rapid automatic event notification service signal 112 includes a link accessible by the medical provider to rapidly access the data of interest from the web-site 70, and which requires a response by the recipient. Such signal 112, and others herein, may also activate an automatic time notated archival feature for later reference for various purposes. As in much of this invention, this path may require a security validation or authentication, depending on system, patient service, and legal requirements. Various biometric or other cipher-like technologies may be utilized, although a smart card or other intelligent and convenient tool may be preferred. For example, one embodiment of a smart card may be able to record medical updates of the patient's health record whenever it is used for access authentication, and thus provide an element of redundancy to the medical record.

First medical provider 106, or others described herein, may be granted secure single sign in rights to facilitate rapid access for the designated individual to the various elements of this system and architecture. This feature may also be transportable as that professional traverses among electronic links in order to ensure connectivity during the response to a detected event, or the access may be a graded access according to an event severity algorithm or other grading technique.

A further feature may include link or other access to a database 118 or other medium having an electronic medical health record 122 of the patient who is the source of the automatic event notification service signal 112. In this manner the patient's pertinent medical history may be presented along with the new data which caused the medical alert, thereby allowing the medical provider with proper perspective and accuracy as to the specific patient. In one embodiment, electronic medical health record 122 is a data rich format such as that known as Extended Markup Language (XML), although various data formats may be utilized to provide the advantages and data contemplated. Moreover, in one embodiment, a single page format is preferred but not required, in order to allow the efficiencies of a template that may be efficiently structured for a single screen display of the most critical information to a final decision maker. This may include a summary, a waveform, a differential diagnosis analysis, coding options, trending graphs, overlays of patient history versus current data, or other high relevance formatted data appropriate to that patient or patient class. Rapid augmenting of that formatted information is also possible, when desired, due to the configuration of this system and the rich data mining capabilities it enables.

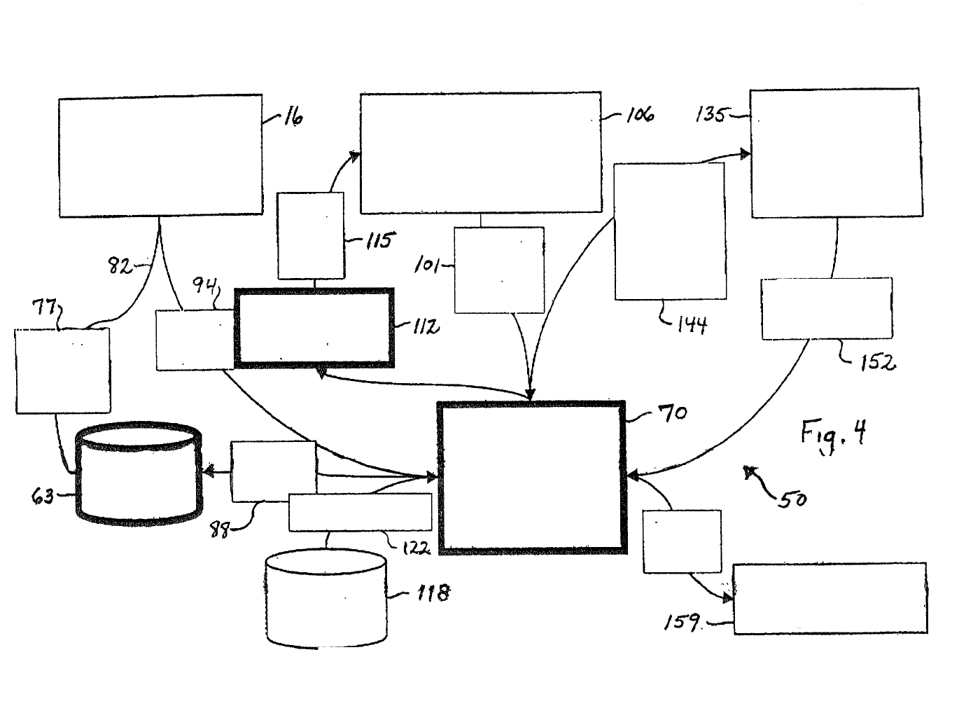

FIG. 4 illustrates a further aspect of the invention in which a second medical provider 135 receives either automatic event notification service signal 112 or, preferably, another form of notification signal 144 which has been generated by another medical provider, such as first medical provider 106. Notification signal 144 is a post-triage type of notification, i.e. after the initial analysis of the high relevance physiologic data has occurred by a first medical provider. The second medical provider may be a more specialized or more highly qualified provider, such as a cardiologist, surgeon or other type of physician. In one embodiment, notification signal may include recommendations or commentary from the first medical provider, and may be in the form of a secure asynchronous XML message, although various formats may be acceptable or preferred, including for example that necessary to enable the above described single page formatted communication. The notification signal 144 may also be formatted for display on a mobile or stationary device of medical provider 135, such as a handheld or other form of personal digital assist device. In this manner, even if the second medical provider is in the process of another activity, it may be possible to have the information observed or otherwise understood from the display and for the second medical 135 to direct appropriate medical care in the form of instructions to the patient and other care givers. In one embodiment it is contemplated for the second medical provider 135 to observe the emergent conditions of patient 16 and then dictate a secure voice message 152 (for example, a WAV file attachment or the like) for transmission back to a web-enabled site, such as web-site 70, for merger with the original data and automatic event notification service signal 112 for subsequent transmission to the patient 16. Of course, this information in the form of a combined signal of both the patient's physiologic data and the medical provider's interpretation and care recommendation may also be transmitted to a proxy or other patient advocate 159, as well as to automatic clinic appointment scheduler prompting mechanisms or the like.

Notification of patient 16 that a health-related message is waiting for review may also be sent to the patient advocate 159, such as a close relative of the patient, to ensure that the patient reviews, understands, and complies with the advice of the medical provider(s). This may be a sequence such as receipt of an electronic mail message urging activation of a link to the web-site 70 with automatic routing to message 152. In addition to the various communication options noted herein above, it is possible to have a dedicated channel, optional pop-up alert channel, webTV-like device, or dedicated internet service or portal available to the patient or patient advocate. This may be a service provided by existing or new communication service providers as a fee subscription or other revenue generating mechanism which is able to emerge and possibly block other communications until cleared by the recipient.

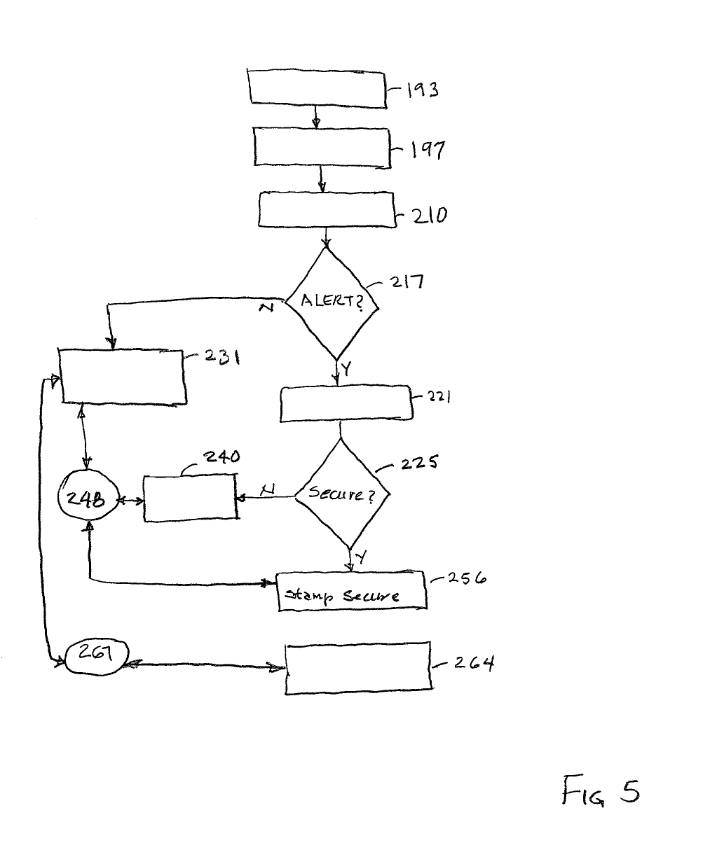

FIG. 5 is a high level logic flow chart representative of one embodiment of the computer implemented steps of the invention and the flow of information. It is recognized that alternate flow paths may be implemented within the scope of this invention while still achieving the novelty, inventiveness, and technical contribution that the inventors are merited. Step 193 represents the detect and store features of the medical device that is monitoring the patient or user of the device. Step 197 represents the interrogate function to query and receive physiologic data from the medical device which is likely implanted within a patient. It is recognized that this function step may be obviated for a device which is external in view of different power management options. Step 203 represents the transmission of data to a remotely located patient management database 210 for storage and processing of the received physiologic data. Decision step 217 represents the analysis and set point computation steps necessary to determine whether the data received comprises particularly high relevance data that necessitates an alert. If an alert is not warranted then at step 219 the data is routed to a web site 231 for instantaneous presentation upon authorized access by either the patient, a proxy or a healthcare professional. If an alert is warranted, then at step 221 a formatting process is implemented, and at step 225 a signal is sent to web-site 231 notification routine causing a non-secure alert signal 240 to be transmitted in a push fashion according to pre-set protocol to healthcare providers. These providers in turn query the web site by rapid return link activation 248 in either a secure or non-secure manner which permits transfer of content rich rendered and formatted signal 240 and/or 256 to the healthcare providers via the web site 231, and with the signal comprising the high relevance physiologic data necessary for analysis and comment/action by the healthcare providers, leading to transfer to the patient and proxy 264 again via the web site at step 267 with such comment/action recommendations riding thereon.

This invention contemplates various systems and various types of methods to enable substantial economic and medical improvements to the business, art and science of healthcare. It is believed that these innovations and technical contributions provide a powerful combination of features and service advantages in view of the previously stated challenges to real-time effective management of certain classes of patient, such as those with chronic cardiac disease, constant infusion of medicines, neurologic stimulator requirements, or others, and that this has not been accomplished or realistically contemplated before by others.

What is claimed:

1. An internet-based method for a service to chronically connect a remote patient to a database network for medical device data exchange and review comprising:
providing a web-site having a user interface wherein the user interface includes a secure sign-in input to access the database network site;
receiving at the web-site inputs associated with a specific medical device and patient;
confirming the identity of the medical device and the patient; and
enabling the patient to access the database to use the service.

2. The method of claim 1 wherein the medical device data exchange and review further comprises at least one of the processes of:
uploading the medical device data into the database; and
accessing the medical device data in the database via a secure web site.

3. The method of claim 1 wherein said website includes a proxy right access scheme to provide privileged access to a patient's data by friends and family.

4. An internet-based method for a service to enable a nurse practitioner to access a secure web-site to respond to a notification of an event relating to a remote patient, the method comprising:
alerting the nurse to an event using an event service; and
enabling the nurse to execute secure access to the patient's database in a single sign-on action.

5. The method of claim 4, wherein said single sign-on action includes authentication to a foreign web-site that is passed over to access the secure patient database.

6. An internet-based method for a service to enable a doctor to access a rendered page about conditions of a patient, the method comprising:
alerting a physician about a patient's condition; and
allowing the physician to access the patient's database and wirelessly pull up a page so that the physician's receipt of a report summarizing the patient's condition is confirmed.

7. The method according to claim 7 wherein a patient management network includes the ability to push out the fully rendered page containing patient information and device information to wireless devices and is represented as a complete page replicating a presentation similar to a website page display.

8. The method according to claim 6 wherein said wireless pull-up includes implication of one of a Microsoft pocket PC technology and equivalent.

9. The method according to claim 6 wherein the physician can transcribe a voice message back to the patient and can optionally copy the message to other professionals.

10. The method according to claim 9 wherein the patient is notified about the physician's message upon the patient's home connect receiving the message.

11. An internet-based method for a service to connect a remote patient's friend or family member to a database network for medical device exchange and review comprising:
identifying a friend or family member authorized to access the database network; and/or
sending notification of a patient alert to said friend or family member.

12. The method according to claim 11 wherein said notification is sent via an E-mail to alert said friend or family member wherein a URL is enclosed in the E-mail to enable secure access of the patient database to the recipient after proper authentication.

13. A computer implemented business method for generating very high relevance personalized patient databases, comprising the steps of:
a. providing a medical device to a patient in which the medical device senses very particular data concerning at least one monitored disease related symptom;
b. enabling the medical device to sense the desired signals and re-transmit the signals electronically to a remote data analysis module; and
c. automatically processing the sensed signals to determine whether health-related action is recommended as a consequence of the sensed signals.

14. The method of claim 13 further comprising the step of automatically alerting at least the patient and a healthcare provider that action is recommended.

15. The method of claim 13 further comprising the step of providing data to a remote healthcare professional.

16. The method of claim 15 further comprising the step of activating a voice message and email format back from the healthcare professional to the patient.

17. A computer implemented method for improved data management in the healthcare industry by increasing patient engagement with recommended healthcare delivery modalities, comprising the steps of:
a. providing an implanted medical device configured for automatic sensing of high relevance biologic data of the patient and transmitting that data, or portions thereof, to an information parser of the healthcare professional;
b. configuring a patient accessible electronic interface to receive signals representative of sensed high relevance biological data of the patient;
c. providing selectively programmable computer implemented rapid interpretations of the sensed high relevance biologic data and, when indicated, electronically sharing with the healthcare professional the details of the sensed high relevance biological data without resort to personal contact or face to face meeting between the healthcare professional and the patient; and
d. providing information flow paths for the healthcare professional to further contribute to the knowledge database and patient engagement by offering the patient and a patient's designated advocate direct information about the high relevance biologic data thereby actively engaging the patient in a highly content rich yet efficient manner.

18. A computer implemented internet-based method for an improved connect and monitoring service to rapidly connect remote persons to a database network for medical device data exchange and analysis, said method being characterized in that it comprises:
providing a web-site having a user interface wherein the user interface includes a secure sign-in input to access a database network site;
receiving at the web-site automatic inputs associated with a specific medical device and user of the device;
automatically confirming the identity of the medical device and the user; and
enabling the user to access the database via the web-site to use the service for real time monitoring of high relevance physiologic data mined from all monitored data of the user.

19. The method of claim 18 wherein said web-site further includes a proxy right access scheme to provide privileged access to a user's data by friends or family as programmed.

20. A computer implemented internet-based method for improved user compliance within a medical patient management system in which the system automatically determines which connection protocols to follow to rapidly connect one or more remote persons to a database network for medical device data exchange and analysis under certain conditions, said method being characterized in that it comprises:
providing a web-site having a user interface wherein the user interface includes a secure sign-in input protocol to access a database network site;
receiving at the web-site automatic inputs associated with a specific medical device and user of the device;
automatically confirming the identity of the medical device and the user; and
performing computer implemented analyses to determine which user groups to rapidly and selectively automatically access the database via the web-site for receipt of high relevance physiologic data mined from all monitored data of the user.

21. The computer implemented internet-based method for improved user compliance of claim 20 further comprising:
alerting a select group of medical providers to an event using an event service; and
enabling the select group of medical providers to execute secure access to the device user's database in a single sign-on action per user in the group.

22. The method of claim 21, wherein said single sign-on action includes authentication to a foreign web-site that is passed over to access the secure device user's database.

23. The method of claim 20 further characterized by computer implemented automatic formatting of automatically processed high relevance data mined from all detected data, and electronically pushing the formatted data to an electronic display of at least one member of a group of medical providers whereby at least one of the group of medical providers selectively provides commentary and then directs a data transmission back via the web site to the user of the medical device, to a designated advocate of the user of the medical device, and, optionally, to another member of a medical providers group.

24. A computer implemented patient management network configured for implementing the method of automatically determining which connection protocols to follow to rapidly connect one or more remote persons to a database network for medical device data exchange and analysis under certain conditions, said network being characterized in that it comprises:
a web site having a user interface wherein the user interface includes a secure sign-in input protocol to access a database network site;
sensing and signal components for providing automatic inputs to the web site associated with a specific medical device and user of the device;
processing routines and module for automatically confirming the identity of the medical device and the user; and
processing routines and module for performing computer implemented analyses to determine which user groups to rapidly and selectively automatically access the database via the web-site for receipt of high relevance physiologic data mined from all monitored data of the user.
